(19) United States
(12) Patent Application Publication
(10) Pub. No.: US 2020/0360095 A1
(43) Pub. Date: Nov. 19, 2020

(54) INTERFACING A SURGICAL ROBOTIC ARM AND INSTRUMENT

(71) Applicant: CMR SURGICAL LIMITED, Cambridge Cambridgeshire (GB)

(72) Inventors: James Oliver Grant, Cambridge, Cambridgeshire (GB); Keith Marshall, Cambridge, Cambridgeshire (GB); Martin McBrien, Cambridge, Cambridgeshire (GB)

(21) Appl. No.: 16/966,363

(22) PCT Filed: Jan. 29, 2019
(86) PCT No.: PCT/GB2019/050232

§ 371 Date: Jul. 30, 2020

(30) Foreign Application Priority Data
Jan. 30, 2018 (GB) 1801531.3

(51) Int. Cl. A61B 34/30; A61B 90/50

(52) U.S. Cl. CPC A61B 34/30; A61B 46/10; A61B 90/50

(57) ABSTRACT

A drive interface structure for interfacing one or more drive assembly interface elements of a surgical robot arm and one or more instrument interface elements of a surgical instrument. The drive interface structure includes a sheet of flexible material for providing a barrier between the one or more instrument interface elements and the one or more drive assembly interface elements; and a rigid rim attached to an outer edge of the sheet of flexible material, the rigid rim configured to engage an interface structure for detachably interfacing the surgical robot arm to the surgical instrument so that when the drive interface structure is engaged with the interface structure the sheet of flexible material covers an aperture of the interface structure through which the one more drive assembly interface elements transfer drive to the one or more instrument interface elements.

### BACKGROUND

It is known to use robots for assisting and performing surgery. FIG. 1 illustrates a typical surgical robot 100 which consists of a base 108, an arm 102, and an instrument 105. The base supports the robot, and is itself attached rigidly to, for example, the operating theatre floor, the operating theatre ceiling or a trolley. The arm extends between the base and the instrument. The arm is articulated by means of multiple flexible joints 103 along its length, which are used to locate the surgical instrument in a desired location relative to the patient. The surgical instrument is attached to the distal end 104 of the robot arm. The surgical instrument penetrates the body of the patient 101 at a port 107 so as to access the surgical site. At its distal end, the instrument comprises an end effector 106 for engaging in a medical procedure.

FIG. 2 illustrates a typical surgical instrument 200 for performing robotic laparoscopic surgery. The surgical instrument comprises a base 201 by means of which the surgical instrument connects to the robot arm. A shaft 202 extends between base 201 and articulation 203. Articulation 203 terminates in an end effector 204. In FIG. 2, a pair of serrated jaws are illustrated as the end effector 204. The articulation 203 permits the end effector 204 to move relative to the shaft 202. It is desirable for at least two degrees of freedom to be provided to the motion of the end effector 204 by means of the articulation.

A surgeon utilises many instruments during the course of a typical laparoscopy operation. For this reason, it is desirable for the instruments to be detachable from and attachable to the end of the robot arm with an ease and speed which enables instruments to be exchanged mid-operation. It is therefore desirable to minimise the time taken and maximise the ease with which one instrument is detached from a robot arm and a different instrument is attached.

The operating theatre is a sterile environment. The surgical robotic system must be sterile to the extent it is exposed to the patient. Surgical instruments are sterilised prior to use in an operation, however the robot arm is not sterilised prior to use. Instead, a sterile drape is placed over the whole of the surgical robot prior to the operation. In this way, the patient is not exposed to the non-sterile surgical robot arm. When exchanging instruments mid-operation, it is desirable for the sterile barrier to be maintained.

### SUMMARY OF THE INVENTION

According to a first aspect of the invention, there is provided a drive interface structure for interfacing one or more drive assembly interface elements of a surgical robot arm and one or more instrument interface elements of a surgical instrument, the drive interface structure comprising: a sheet of flexible material for providing a barrier between the one or more instrument interface elements and the one or more drive assembly interface elements; and a rigid rim attached to an outer edge of the sheet of flexible material, the rigid rim configured to engage an interface structure for detachably interfacing the surgical robot arm to the surgical instrument so that when the drive interface structure is engaged with the interface structure the sheet of flexible material covers an aperture of the interface structure through which the one more drive assembly interface elements transfer drive to the one or more instrument interface elements.

The flexible material may comprise one or more drive transfer elements for transferring drive between the one or more instrument interface elements and the one or more drive assembly interface elements.

The one or more drive transfer elements may be linearly displaceable with respect the rigid rim.

Each of the one or more drive transfer elements may comprise a cup-shaped portion for receiving a drive assembly interface element or an instrument assembly element.

Each of the one or more drive transfer elements may comprise a rim portion surrounding the cup-shaped portion.

The one or more drive transfer elements may be formed of a more resilient material than the flexible material.

The one or more drive transfer elements may be formed of a plastic material.

The drive interface structure may further comprise one or more fasteners supported by the rigid rim and protruding from a bottom surface of the rigid rim, the one or more fasteners configured to engage the surgical robot arm so as to retain the drive interface structure to the surgical robot arm

The drive interface structure may further comprise one or more fasteners supported by the rigid rim and protruding from a bottom surface of the rigid rim, the one or more fasteners configured to engage the interface structure so as to retain the drive interface structure to the interface structure.

The one or more fasteners may be configured to extend through the aperture of the interface structure when the drive interface structure engages the interface structure.

The drive interface structure may further comprise a gasket on a bottom surface of the rigid rim, the gasket configured to form a tight seal around the aperture when the drive interface structure engages the interface structure.

The rigid rim may be configured to engage a gasket surrounding the aperture so as to form a tight seal around the aperture when the drive interface structure engages the interface structure.

The rigid rim may comprise one or more protrusions which are configured to engage the surgical instrument when the surgical instrument is brought into engagement with the drive interface structure so as to clamp the seal around the aperture when the drive interface structure engages the interface structure.

At least one of the one or more protrusions may be situated on a corner of the rigid rim.

At least one of the one or more protrusions may extend along a rear edge of the rigid rim.

The rigid rim may comprise one or more flanges configured to conceal one or more drive interface engagement markings on the interface structure when the drive interface structure is engaged with the interface structure.

The sheet of flexible material may be bonded to the rigid rim.

According to a first aspect of the invention, there is provided an interface structure for detachably interfacing a surgical robot arm to a surgical instrument, the interface structure comprising: a base portion comprising a rim surrounding an aperture through which one or more drive assembly interface elements of the surgical robot arm transfer drive to one or more instrument interface elements of the surgical instrument, the rim comprising a first surface for facing the surgical instrument and a second surface for facing the surgical robot arm; and one or more protrusions supported by the base portion and protruding from the first surface, the one or more protrusions configured to receive a drive interface structure comprising a sheet of flexible material and align the drive interface structure so that the flexible material of the drive interface structure covers the aperture.

The first surface may comprise one or more drive interface engagement markings that are visible when the drive interface structure is not engaged with the interface structure and that are visible when the drive interface structure is engaged with the interface structure.

The interface structure may further comprise a plurality of second fasteners supported by the base portion and protruding from the first surface, the plurality of second fasteners configured to engage the surgical instrument so as to retain the interface structure to the surgical instrument.

The plurality of second fasteners may be shaped so as to, when the surgical instrument is attached to the interface structure, restrain the surgical instrument from moving relative to the interface structure in directions perpendicular to the axial direction of the surgical robot arm.

The plurality of second fasteners may be shaped so as to, when the surgical instrument is attached to the surgical robot arm, restrain the surgical instrument from moving relative to the surgical robot arm in an axial direction of the surgical robot arm.

When the interface structure is attached to the surgical robot arm, the base portion may be parallel to the axial direction of the surgical robot arm.

The interface structure may further comprise a rear wing portion attached to a rear edge of the rim of the base portion, the rear wing portion configured to cover a proximal exposed surface of the surgical robot arm.

The rear wing portion may comprise one or more first fasteners for fastening to the proximal exposed surface of the surgical robot arm.

The interface structure may further comprise a front wing portion attached to a front edge of the rim of the base portion, the front wing portion configured to cover a distal exposed surface of the surgical robot arm.

The interface structure may further comprise an envelope portion which connects opposing edges of the base portion so as to, when engaged on the surgical robot arm, retain the interface structure to the surgical robot arm.

The envelope portion may be shaped so as to, when the interface structure is engaged on the surgical robot arm, circumscribe an exterior surface of the surgical robot arm.

The base portion and the envelope portion may be integrally formed.

The rim may have an opening which is configured to receive a portion of the instrument, the opening being valley-shaped with valley walls.

According to a third aspect of the invention, there is provided an interface assembly comprising: the drive interface structure of the first aspect and the interface structure of the second aspect.

Where the interface structure has a rear wing portion with first fasteners the interface assembly may further comprise a locking element moveably mounted on the rear wing portion, the locking element moveable between a locked position and an unlocked position, when the locking element is the locked position the locking element biases the plurality of first fasteners to an engaged position and when the locking element is in the unlocked position the locking element does not bias the first fasteners to the engaged position.

The locking element may terminate in a drape.

The drape and the locking element may be integrally formed.

According to a fourth aspect of the invention, there is provided a surgical robot arm for use in robotic surgery, the surgical robot arm comprising: a base; and a series of articulations connecting the base to an interfacing portion at the distal end of the surgical robot arm, the series of articulations enabling the interfacing portion to be articulated relative to the base; and the interfacing portion configured to interface a surgical instrument by retaining the interface structure of the second aspect.

### BRIEF DESCRIPTION OF THE FIGURES

The present invention will now be described by way of example with reference to the accompanying drawings. In the drawings:

FIG. 1 illustrates a surgical robot performing a surgical procedure;

FIG. 2 illustrates a known surgical instrument;

FIG. 3 illustrates a surgical robot;

FIG. 4 illustrates a top view of an instrument interface;

FIG. 5 illustrates a bottom view of the instrument interface of FIG. 4;

FIG. 6 illustrates a drive assembly interface of a robot arm;

FIG. 7 illustrates an instrument being positioned into engagement with a robot arm via an interface assembly comprising an interface structure, a locking element and a drive interface structure;

FIG. 8 illustrates the interface structure and the locking element of FIG. 7 when the locking element is in the unlocked position;

FIG. 9 illustrates the interface structure and the locking element of FIG. 7 when the locking element is in the locked position;

FIG. 10 illustrate a top view of the drive interface structure of FIG. 7 in isolation;

FIG. 11 illustrates a bottom view of the drive interface structure of FIG. 10;

FIG. 12 is a cross-sectional view along the longitudinal axis of the interface structure of an instrument engaged with the drive interface structure of FIG. 9 and the interface structure of claim 8 (without the locking element), which are engaged with a robot arm;

FIG. 13 is a cross-sectional view along the longitudinal axis of the interface structure of the drive interface structure of FIG. 9 engaged with the interface structure of claim 8 (without the locking element); and

FIG. 14 illustrates an instrument attached to a robot arm via the interface assembly of FIG. 7.

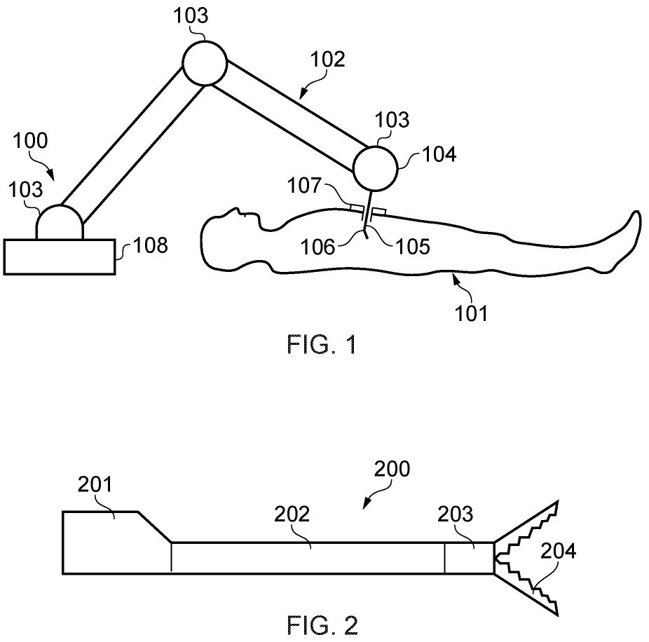

### DETAILED DESCRIPTION

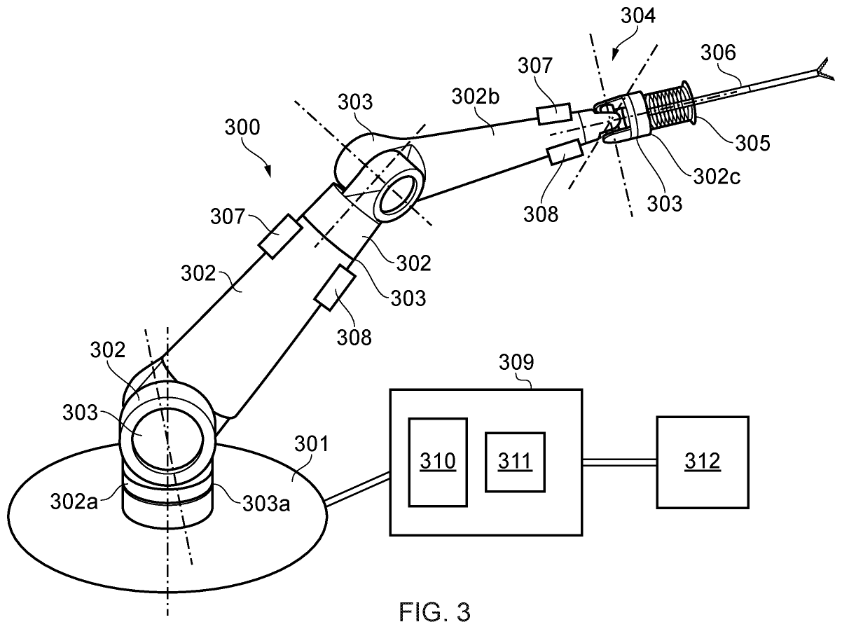

FIG. 3 illustrates a surgical robot having an arm 300 which extends from a base 301. The arm comprises a number of rigid limbs 302. The limbs are coupled by revolute joints 303. The most proximal limb 302a is coupled to the base by joint 303a. It and the other limbs are coupled in series by further ones of the joints 303. Suitably, a wrist 304 is made up of four individual revolute joints. The wrist 304 couples one limb (302b) to the most distal limb (302c) of the arm. The most distal limb 302c carries an attachment 305 for a surgical instrument 306. Each joint 303 of the arm has one or more motors 307 which can be operated to cause rotational motion at the respective joint, and one or more position and/or torque sensors 308 which provide information regarding the current configuration and/or load at that joint.

Suitably, the motors are arranged proximally of the joints whose motion they drive, so as to improve weight distribution. For clarity, only some of the motors and sensors are shown in FIG. 3. The arm may be generally as described in our co-pending patent application PCT/GB2014/053523.

The arm terminates in an attachment 305 for interfacing with the instrument 306. Suitably, the instrument 306 takes the form described with respect to FIG. 2. The instrument has a diameter less than 8 mm. Suitably, the instrument has a 5 mm diameter. The instrument may have a diameter which is less than 5 mm. The instrument diameter may be the diameter of the shaft. The instrument diameter may be the diameter of the profile of the articulation. Suitably, the diameter of the profile of the articulation matches or is narrower than the diameter of the shaft. The attachment 305 comprises a drive assembly for driving articulation of the instrument. Movable interface elements of the drive assembly interface mechanically engage corresponding movable interface elements of the instrument interface in order to transfer drive from the robot arm to the instrument. One instrument is exchanged for another several times during a typical operation. Thus, the instrument is attachable and detachable from the robot arm during the operation. Features of the drive assembly interface and the instrument interface aid their alignment when brought into engagement with each other, so as to reduce the accuracy with which they need to be aligned by the user.

The instrument 306 comprises an end effector for performing an operation. The end effector may take any suitable form. For example, the end effector may be smooth jaws, serrated jaws, a gripper, a pair of shears, a needle for suturing, a camera, a laser, a knife, a stapler, a cauteriser, a suctioner. As described with respect to FIG. 2, the instrument comprises an articulation between the instrument shaft and the end effector. The articulation comprises several joints which permit the end effector to move relative to the shaft of the instrument. The joints in the articulation are actuated by driving elements, such as cables. These driving elements are secured at the other end of the instrument shaft to the interface elements of the instrument interface. Thus, the robot arm transfers drive to the end effector as follows: movement of a drive assembly interface element moves an instrument interface element which moves a driving element which moves a joint of the articulation which moves the end effector.

Controllers for the motors, torque sensors and encoders are distributed with the robot arm. The controllers are connected via a communication bus to control unit 309. A control unit 309 comprises a processor 310 and a memory 311. Memory 311 stores in a non-transient way software that is executable by the processor to control the operation of the motors 307 to cause the arm 300 to operate in the manner described herein. In particular, the software can control the processor 310 to cause the motors (for example via distributed controllers) to drive in dependence on inputs from the sensors 308 and from a surgeon command interface 312. The control unit 309 is coupled to the motors 307 for driving them in accordance with outputs generated by execution of the software. The control unit 309 is coupled to the sensors 308 for receiving sensed input from the sensors, and to the command interface 312 for receiving input from it. The respective couplings may, for example, each be electrical or optical cables, or may be provided by a wireless connection. The command interface 312 comprises one or more input devices whereby a user can request motion of the end effector in a desired way. The input devices could, for example, be manually operable mechanical input devices such as control handles or joysticks, or contactless input devices such as optical gesture sensors. The software stored in memory 311 is configured to respond to those inputs and cause the joints of the arm and instrument to move accordingly, in compliance with a pre-determined control strategy. The control strategy may include safety features which moderate the motion of the arm and instrument in response to command inputs. Thus, in summary, a surgeon at the command interface 312 can control the instrument 306 to move in such a way as to perform a desired surgical procedure. The control unit 309 and/or the command interface 312 may be remote from the arm 300.

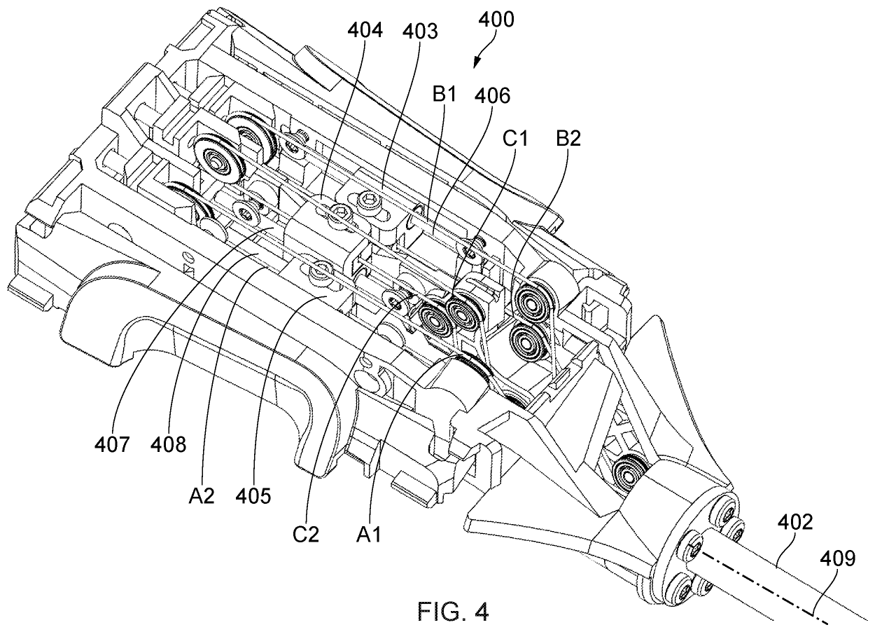

FIGS. 4 to 6 illustrate an exemplary mechanical interconnection of the drive assembly interface and the instrument interface in order to transfer drive from the robot arm to the instrument. The shaft 402 of the instrument terminates in the instrument interface 400. The instrument interface 400 comprises a plurality of instrument interface elements 403, 404, 405. Pairs of driving elements (A1, A2), (B1, B2), (C1, C2) extend into the instrument interface 400 from the end of the shaft 402. Each pair of driving elements terminates in one of the instrument interface elements. In the example shown in FIG. 4: driving element pair A1, A2 terminates in instrument interface element 405; driving element pair B1, B2 terminates in instrument interface element 403; and driving element pair C1, C2 terminates in instrument interface element 404.

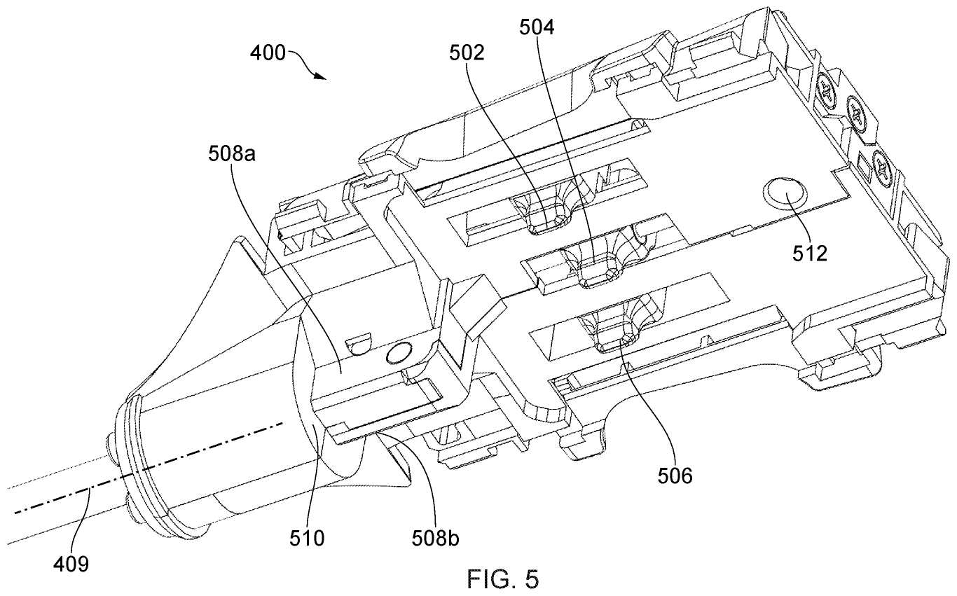

FIGS. 4 and 5 illustrate three instrument interface elements and three driving element pairs. In other examples, there may be greater than or fewer than three instrument interface elements. There may be greater than or fewer than three driving element pairs. In FIGS. 4 and 5 there is a one-to-one relationship between instrument interface elements and driving element pairs. In other examples, there may be any other coupling relationship between the instrument interface elements and driving element pairs. For example, a single instrument interface element may drive more than one pair of driving elements. In another example, more than one instrument interface element may drive a single pair of driving elements.

The instrument interface elements are displaceable within the instrument interface. In the example shown, the instrument interface elements are slideable along rails. Instrument interface element 403 is slideable along rail 406, instrument interface element 404 is slideable along rail 407, and instrument interface element 405 is slideable along rail 408. Each instrument interface element is displaceable along a direction parallel to the direction of elongation of the pair of driving elements which that instrument interface element holds captive. Each instrument interface element is displaceable in a direction substantially parallel to the longitudinal axis 409 of the instrument shaft 402. When the instrument interface element moves along its rail, it causes a corresponding movement to the driving element pair secured to it. Thus, moving an instrument interface element drives motion of a driving element pair and hence motion of a joint of the instrument.

In the example of FIGS. 4 and 5, each instrument interface element comprises a fin 502, 504, 506 (FIG. 5) which is the portion of the instrument interface element which engages the drive assembly interface element.

In another example, each drive assembly interface element comprises a fin, and each instrument interface element comprises a socket for receiving the fin of the corresponding drive assembly interface element.

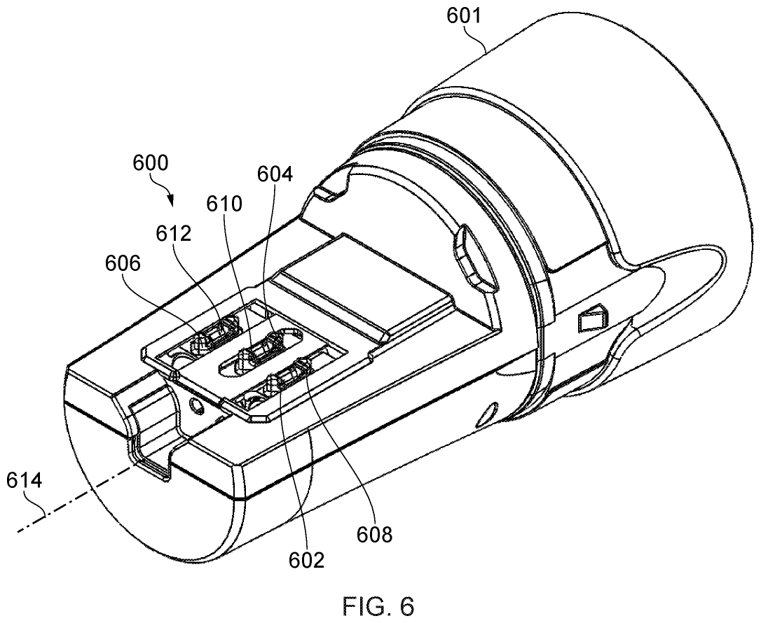

FIG. 6 illustrates an exemplary drive assembly interface 600 at the end of a robot arm 601. Drive assembly interface 600 mates with instrument interface 400. Drive assembly interface 600 comprises structure for receiving the instrument interface elements of the instrument interface of FIGS. 4-5. Specifically, drive assembly interface elements 602, 604, 606 receive instrument interface elements 403, 404, 405. In the example shown, each drive assembly interface element comprises a socket for receiving the fin 502, 504, 506 of the corresponding instrument interface element. Socket 608 of drive assembly interface element 602 receives fin 502 of instrument interface element 403. Socket 610 of drive assembly interface element 604 receives fin 504 of instrument interface element 404. Socket 612 of drive assembly interface element 606 receives fin 506 of instrument interface element 405.

FIG. 6 illustrates three drive assembly interface elements. In other examples, there may be greater than or fewer than three drive assembly interface elements. In FIGS. 4-6 there is a one-to-one relationship between instrument interface elements and drive assembly interface elements. In other examples, there may be any other coupling relationship between the instrument interface elements and drive assembly interface elements. For example, a single drive assembly interface element may drive more than one instrument interface element. In another example, more than one drive assembly interface element may drive a single instrument interface element.

Each drive assembly interface element is displaceable within the drive assembly. This displacement is driven. For example, the displacement may be driven by a motor and lead screw arrangement. In the example shown, the drive assembly interface elements are slideable along rails. Each drive assembly interface element is displaceable along a direction parallel to the longitudinal axis 614 of the terminal link of the robot arm. When the drive assembly interface element moves along its rail, it causes a corresponding movement to the instrument interface element that it holds captive. Thus, driving motion of a drive assembly interface element drives motion of an instrument interface element which drives articulation of the end effector of the instrument.

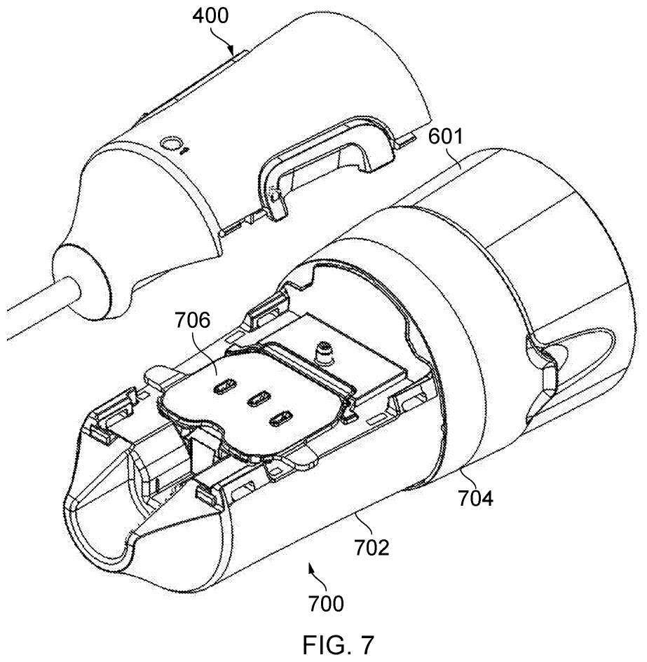

FIG. 7 illustrates the instrument being placed into engagement with the robot arm. When instrument interface element 403 and drive assembly interface element 602 are engaged, instrument interface element 404 and drive assembly interface element 604 are engaged, and instrument interface element 405 and drive assembly interface element 606 are engaged, the instrument interface elements and the drive assembly interface elements are all displaceable in the same direction. This direction is parallel to both the longitudinal axis 614 of the terminal link of the robot arm and the longitudinal axis 409 of the instrument shaft 402.

During an operation, the surgical robot is shrouded in a sterile drape to provide a sterile barrier between the non-sterile surgical robot and the sterile operating environment. The surgical instrument is sterilised before being attached to the surgical robot. The sterile drape is typically constructed of a plastic sheet, for example made of polyester, polypropylene, polyethylene or polytetrafluoroethylene (PTFE). Suitably, the drape is flexible and/or deformable.

The sterile drape does not pass directly between the drive assembly interface 600 and the instrument interface 400. An interface assembly 700 is attached to the drape for interfacing between the drive assembly interface 600 and the instrument interface 400. The exemplary interface assembly 700 comprises an interface structure 702 which engages the robot arm and the instrument so as to retain the interface element to the robot arm and instrument respectively, and a removable drive interface structure 706 comprising flexible material for interfacing between the instrument interface elements 403, 404, 405 and the drive assembly interface elements 602, 604, 606. In some cases, the interface assembly 700 may also include a moveable locking element 704 mounted on the interface structure 702 for locking the interface structure 702 to the robot arm 601.

Suitably the drape is attached to the locking element 704. However, in other examples the drape may be attached to the interface structure 702. The interface structure 702 or the locking element 704 may be integrally formed with the drape. Alternatively, the interface structure 702 or the locking element 704 may be formed separately from the drape and subsequently attached to the drape. Either way, the interface structure 702 and the locking element 704 are sterile. One side of the interface structure 702 directly contacts the drive assembly interface. Another side of the interface structure 702 directly contacts the instrument interface. As described in more detail below, the interface structure 702 comprises a rim surrounding an aperture through which the drive assembly interface elements transfer drive to the interface assembly elements. The drive interface structure 706 is also sterile and comprises a flexible material surrounded by a rigid rim. The drive interface assembly 706 is configured to engage the interface structure 702 so that the flexible material provides a sterile barrier between the drive assembly interface elements and the instrument interface elements. Thus, the interface structure 702 and drive interface structure 706 together prevent the non-sterile drive assembly interface from directly touching the sterile instrument interface and hence maintain the sterile barrier between the surgical robot and the surgical instrument.

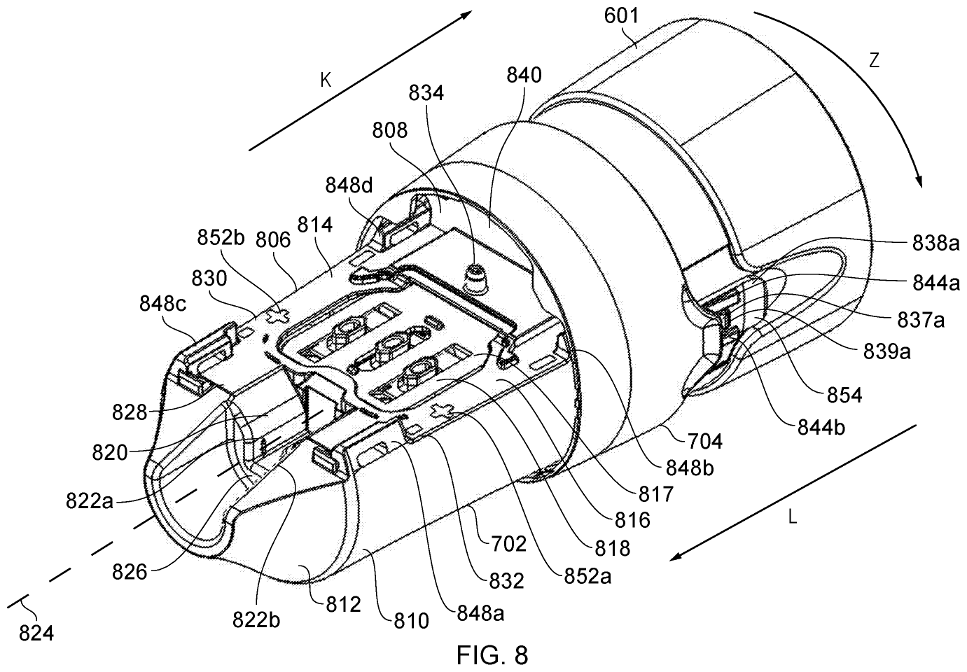

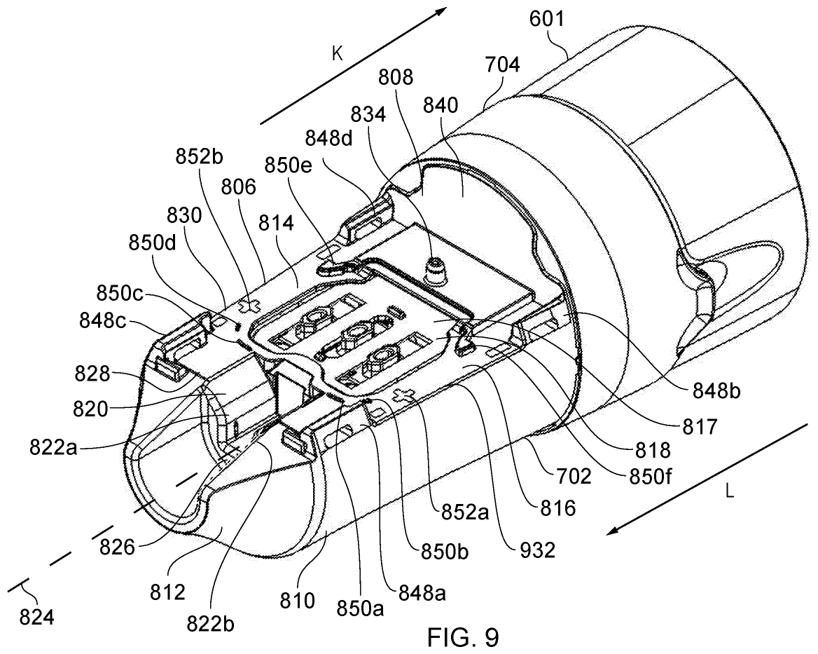

FIGS. 8 and 9 show an exemplary interface structure 702 and locking element 704 attached to a robot arm 601. Suitably, the interface structure 702 is a single moulded part. The interface structure 702 comprises a base portion 806, means for engaging the surgical robot so as to retain the interface structure to the robot arm, and means for engaging the surgical instrument so as to retain the interface structure to the robot arm. Suitably, when the interface structure 702 is attached to the surgical robot arm, the base portion 806 lies parallel to the axial direction of the terminal link of the robot arm. The base portion 806 comprises a first surface 814 which faces the surgical instrument when the instrument is attached to the robot arm. Specifically, the first surface 814 faces the instrument interface 400. The base portion 806 comprises a second surface (not visible in FIGS. 8-9) which opposes the first surface 814. The second surface faces the robot arm when the instrument is attached to the robot arm. Specifically, the second surface faces the drive assembly interface 600. The first surface 814 may be flat. The second surface may be flat.

The base portion 806 of the interface structure 702 comprises a rim 816 surrounding an aperture 817 that leads to a hollow interior 818. When the interface structure 702 is attached to the robot arm, the rim 816 is encompassed within a boundary formed by the external surface of the surgical robot arm in the longitudinal direction of the surgical robot arm. The rim 816 has an opening 820 which receives the portion of the chassis of the instrument interface 400 into which the end of the shaft of the surgical instrument terminates. The opening 820 is valley-shaped, with valley walls 822a and 822b. The instrument interface 400 has contact faces 508a and 508b, which have a complementary shape to the valley walls 822a, 822b such that contact faces 508a, 508b engage valley walls 822a, 822b when the instrument interface is engaged in the interface structure. Suitably, the contact faces 508a, 508b have a light interference fit to the valley walls 822a, 822b. The engagement of the contact faces and valley walls acts to prevent motion of the instrument parallel to the base portion 806 and transverse to the longitudinal axis 824 of the interface structure 702. The engagement of the contact faces and valley walls acts to prevent rotational motion of the instrument about the longitudinal axis 824 of the interface structure 702. The instrument interface 400 suitably has a contact face 510 which abuts a contact face 826 of the opening 820 when the instrument interface is engaged in the interface structure. The contact face 826 is transverse to the longitudinal axis 824. The contact face 826 is parallel to the front edge 828 of the base portion. The front edge 828 joins the two longer outer edges 830 and 832 of the base portion.

The base portion 806 may comprise a pin 834. Pin 834 may be located on the longitudinal axis 824 of the interface structure 702. Instrument interface 400 may comprises a recess 512 into which the pin 834 of the interface structure 702 engages. Recess 512 may be located on the longitudinal axis 409 of the instrument interface 400. The pin 834 fits snugly into the recess 512.

The engagement of the pin 834 in the recess 512, and the bearing of the contact face 510 on the contact face 826 cause the instrument to be aligned axially with the interface structure. They also act to restrain movement of the instrument relative to the interface structure in the axial direction.

The hollow interior 818 receives the instrument interface elements which engage with the drive assembly interface elements via the aperture 817. Specifically either the drive assembly interface elements or the instrument interface elements, or both, are received in the aperture 817 to enable the drive assembly interface elements to engage the instrument interface elements to provide drive to the instrument interface elements. As is described in more detail below, the drive assembly interface elements do not directly contact the instrument interface elements as the instrument interface elements are sterile and the drive assembly interface elements are not sterile. In contrast, the drive assembly interface element engage the instrument interface elements via a sterile flexible material that is removably engaged with the interface structure 702.

The interface structure 702 may also comprise a rear portion 808 attached to the rear edge 836 of the rim 816 of the base portion. Both the rear edge 836 and the front edge 828 join the two longer outer edges 830 and 832 of the base portion 806. When the interface structure 702 is attached to the robot arm, the front edge 828 is positioned closer to the free distal end of the robot arm than the rear edge 836. The rear portion 808 comprises one or more first fasteners 838a, 838b for fastening the rear portion 808 to a proximal exposed surface of the robot arm. In the example shown in FIGS. 8-9, there are two first fasteners 838a, 838b which are each in the form of a clip with an aperture 837a, 837b which is configured to clip onto a lug 839a (only one is shown) of a slip collar of the robot arm. Any suitable fastener may be used for the first fastener. The rear portion 808 may be integrally moulded with the base portion 806.

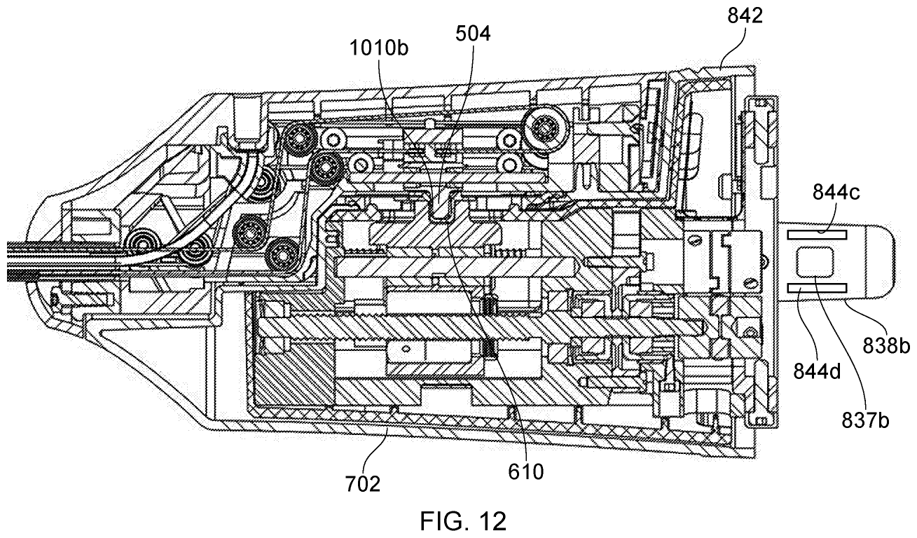

In the example of FIGS. 8-9 the rear portion 808 comprises a rear wing portion 840 and a collar 842 (sees FIG. 12, 13). The rear wing portion 840 is attached to the rear edge 836 of the rim 816 of the base portion 806. The rear wing portion 840 covers a proximal exposed surface of the robot arm. Rear wing portion 840 is suitably shaped to match the shape of the proximal exposed surface of the robot arm. Suitably, the rear wing portion 840 is angled relative to a direction perpendicular to the base portion 806 away from the distal end of the robot arm, by an angle α. For example, 20°<α<50°, or 30°<α<40°, or 35°<α<37°. Where the interface structure 702 is attached to the drape this aids the process of passing the drape over the robot arm.

The collar 842 is attached to the rear wing portion 840. Suitably, the collar 842 is integrally moulded with the rear wing portion 840. In the example of FIGS. 8-9, the collar has substantially cylindrical inner surface. The collar 82 may have a substantially frustoconical outer surface. The collar 842 may terminate in a sterile drape (not shown). Alternatively the locking element 704 (described in more detail blow) may terminate in a sterile drape (not shown). In either case the sterile drape shrouds the surgical robot arm. In practice, the interface assembly 700 is installed axially onto the robot arm, i.e. along the longitudinal axis of the drive assembly interface at the end of the robot arm. The drape is then unravelled down the robot arm.

The interface structure 702 may further comprise an envelope portion 810 that connects the longer of the outer edges 830, 832 of the base portion 806. These longer edges are those which run down the length of the base portion 806. As shown in FIGS. 8-9, the envelope portion 810 may circumscribe the drive assembly. The shape of the envelope portion 810 may match the shape of the outer surface of the robot arm at the drive assembly. Suitably, the envelope portion 810 contacts the exterior surface of the robot arm at the drive assembly. This contact may be a snug fit. This contact may be sheath-like. In this way, the envelope portion 810 bears on the exterior surface of the robot arm at the drive assembly. The envelope portion 810 thereby acts to retain the interface structure 702 to the robot arm. Specifically, the envelope portion 810 acts to retain the interface structure 702 to the robot arm in directions transverse to the longitudinal axis 824 of the interface structure 702.

The interface structure 702 may further comprise a front wing portion 812. Front wing portion 812 is attached to the front edge 828 of the rim 816 of the base portion 806. Suitably, the front wing portion 812 is integrally moulded with the base portion and envelope portion of the interface structure. The front wing portion 812 covers a distal exposed surface of the surgical robot arm. The inner surface of the front wing portion 812 may be shaped to match the shape of the distal exposed surface of the robot arm. The outer surface of the front wing portion 812 may be shaped to match the shaped of the front of the instrument.

The base portion 806 may support a plurality of second fasteners 848a, 848b, 848c, 848d for engaging the surgical instrument so as to retain the surgical instrument to the interface structure. These second fasteners 848a, 848b, 848c, 848d protrude from the first surface 814 of the base portion 806 transverse to the first surface 814. The second fasteners 848a, 848b, 848c, 848d protrude from the longer of the outer edges 830, 832 of the base portion 806.

These longer edges are those which run down the length of the base portion 806. These longer edges are those which connect the first surface and the side flange portions. In the example of FIGS. 8-9, there are four second fasteners. There may, however, be more than four, or fewer than four, second fasteners. Preferably, there are at least two second fasteners, one on either longer outer edge 830, 832, in order to prevent the instrument from dislodging from the interface assembly in a direction perpendicular to the longitudinal axis 824 of the interface structure 702. In other words, to prevent the instrument from dislodging from the interface structure in a direction perpendicular to the longitudinal axes 614 and 409 of the terminal link of the robot arm and the instrument shaft respectively, when the instrument is engaged with the robot arm.

The second fasteners 848a, 848b, 848c, 848d may be integrally formed with the base portion 806.

Each second fastener may be shaped such that when the surgical instrument is attached to the interface structure, the second fasteners restrain the surgical instrument from moving relative to the interface structure along the direction of the longitudinal axis 824 of the interface structure 702 away from the front wing portion 812. That is towards the surgical robot arm when the interface structure is attached to the surgical robot arm.

When the instrument is interfaced to the robot arm, via the interface assembly 700, the performance of the instrument is linked to the quality of the connection of the instrument, via the interface assembly 700, to the arm. One measure of the quality of the connection is the ability of the instrument interface, and thus the instrument, to be retained on the arm in the right place. Accordingly, the interface assembly 700 may also comprise a locking element 704 to lock the interface structure 702 to the robot arm. By ensuring that the interface assembly 700, and thus the instrument, are retained on the arm in the right place during use the locking element 704 may improve performance of the instrument.

The locking element 704 is moveably mounted to the rear portion 808 of the interface structure. The locking element 704 is moveable between an unlocked position (FIG. 8) and a locked position (FIG. 9). In the locked position the locking element 704 biases the first fasteners 838a, 838b towards an engaged position (e.g. inwards) to lock the interface structure 702 onto the robot arm. That is, the locking element biases the first fasteners 838a, 838b towards the surgical robot arm when the interface assembly 700 is attached to the surgical robot arm. In contrast, in the unlocked position the locking element 704 does not bias the first fasteners 838a, 838b towards an engaged position (e.g. towards the surgical robot arm when the interface assembly is attached to the surgical robot arm). As shown in FIGS. 8-9, the locking element 704 may have a substantially cylindrical inner surface wherein the diameter of the cylinder is larger than the diameter of the collar 842 so that the locking element 704 can be mounted on the outside of the collar 842. The locking element 704 may be frustoconical. However, in other examples, the locking element 704 may take a different shape so long as the locking element is able to provide significant pressure on the first fasteners 838a, 838b to ensure that the first fasteners 838a, 838b don't come dislodged from the robot arm (e.g. the lugs 839a, 839b described below) during use. For example, where the locking element is made of a stiff material, such as, but not limited to fibre reinforced plastic and magnesium (e.g. similar to the used in camera bodies), the locking element may alternatively have a semi-circle cross section.

In the example of FIGS. 8-9 the locking element 704 is slideable along the collar 842 in a direction parallel to the longitudinal axis of the interface structure to move between the unlocked position and the locked position. Specifically, the locking element is slideable in direction K to move from the unlocked position to the locked position and is slideable in direction L to move from the locked position to the unlocked position. In this example, as the locking element 704 slides into the locked position the locking element 704 engages the first fasteners 838a, 838b and pushes the first fasteners 838a, 838b towards an engaged position (e.g. inward). That is, the locking element 704 pushes the first fasteners towards the robot arm and onto the lugs 839a, 839b of the robot arm when the interface assembly is attached to the surgical robot arm. In other examples the locking element 704 may be rotatable about the collar 842 (e.g. in direction Z) to move between the unlocked position and the locked position.

The first fasteners 838a, 838b may comprise one or more sloped protrusions or ramps 844a, 844b, 844c, 844d on an outer surface 854 of the first fasteners 838a, 838b which aid the locking element 704 in biasing the first fasteners 838a, 838b inward (e.g. toward the robot arm). Advantageously the depth of the sloped protrusions 844a, 844b, 844c, 844d increases in the direction in which the locking element 704 moves into the locking position so that as the locking element 704 moves into the locked position the pressure the locking element 704 applies to the first fasteners 838a, 838b increases. For example, in FIGS. 8-9 the locking element 704 is slideable in direction K (FIG. 8) to move from the unlocked position to the locked position thus the depth of the sloped protrusions increases in direction K. In contrast, where the locking element 704 is rotatable in direction Z, for example, to move from the unlocked position to the locked position the depth of the sloped protrusions may increase in direction Z.

In the example shown in FIGS. 8-9, each first fastener 838a, 838b comprises two sloped protrusions 844a, 844b, 844c, 844d one on either side of the aperture 837a, 837b of the first fastener 838a, 838b. This allows the locking element 704 to apply substantially equal pressure across the first fasteners 838a, 838b when in the locked position to ensure the first fasteners 838a, 838b remain attached to the robot arm during use. In contrast, if the first fastener 838a of FIGS. 8-9 had only a single sloped protrusion 844a, for example, then the bottom of the first fastener 838a may be held tight to the robot arm, but the top of the first fastener 838a may not be held as tight allowing the top portion of the first fastener 838a to slip off the lug 839a. However, in other examples, there may be more than two, or fewer than two, sloped protrusions 844a, 844b, 844c, 844d per first fastener 838a, 838b. For example, instead of having a single aperture that engages a single lug, the first fasteners may comprise two apertures that engage two different lug portions. In these examples, each first fastener may comprise a single sloped protrusion situated between the two apertures.

In other examples, instead of the sloped protrusions being on an outer surface of the first protrusions the one or more sloped protrusions may be on an inner surface of the locking element. In these cases the depth of the sloped protrusion may decrease in the direction in which the locking element 704 moves into the locking position so that as the locking element 704 moves into the locked position the pressure the locking element 704 applies to the first fasteners 838a, 838b increases.

When the surgical instrument is detached from the surgical robot arm, the interface assembly 700 is retained in the surgical robot arm. The interface assembly is more securely attached to the surgical robot arm than the instrument interface elements are to the drive assembly interface elements. Thus, the interface assembly 700 and the drape, remain attached to the surgical robot arm during instrument exchange. This is important in order to reduce the time taken to change instruments, since the interface structure does not need to be re-attached to the robot arm following detachment of an instrument. It is also important in order to reduce the likelihood of the drape tearing when changing instruments, which would cause the sterile operating environment to become contaminated with the non-sterile environment on the robot arm side of the drape. In the example of FIGS. 8-9, this is achieved by the shape of the envelope portion 810 surrounding the exterior of the robot arm at the drive assembly, and by the envelope portion 810 connecting to the base portion 806 wholly along the length of the sides 830 and 832. When a force is applied transverse to the base portion 806, away from the surgical robot arm, the envelope portion 810 is more resistant to breaking and hence dislodging from its retained position relative to the surgical robot arm than the instrument interface elements are from dislodging from their retained positions in the drive assembly interface elements.

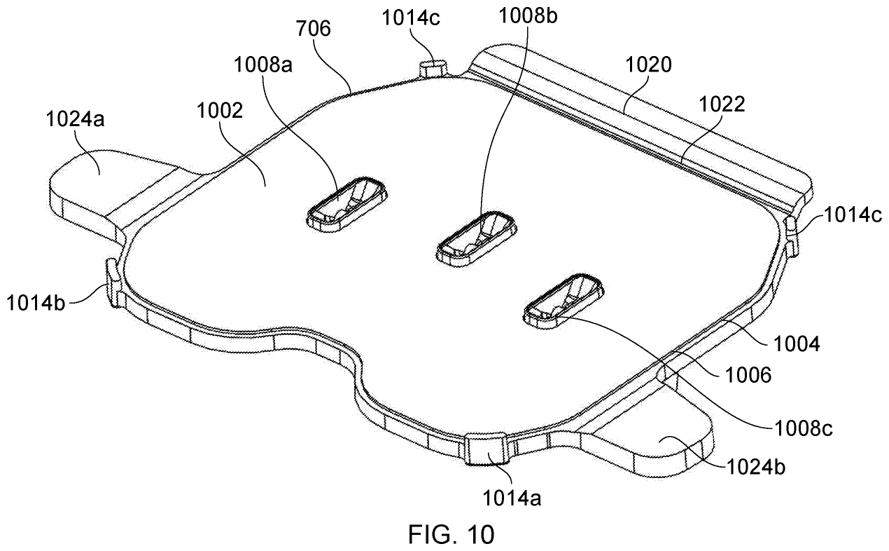

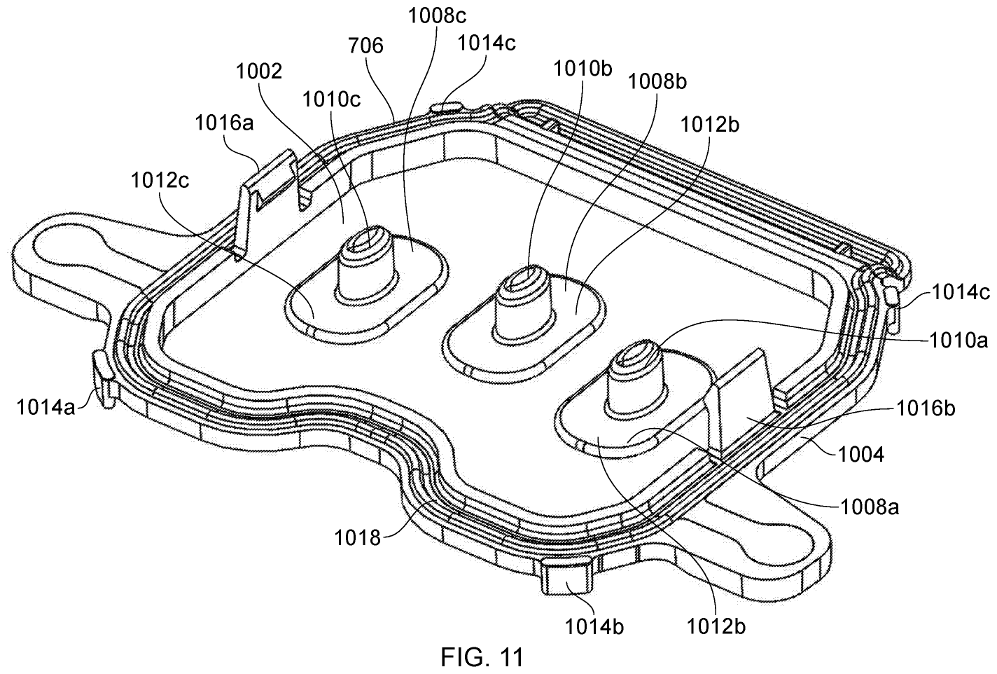

Reference is now made to FIGS. 10 and 11 which illustrate an exemplary drive interface structure 706 in isolation. FIG. 10 is a top view of the drive interface structure 706 and FIG. 11 is a bottom view of the drive interface structure 706.

The drive interface structure 706 comprises a sheet of flexible material 1002 for providing a sterile barrier between the instrument interface elements and the drive assembly interface elements, and a rigid rim 1004 attached to an outer edge 1006 of the sheet of flexible material 1002. The rigid rim 1004 is configured to removably engage the interface structure 702 so that when the drive interface structure is engaged with the interface structure the sheet of flexible material 1002 covers the aperture 817 of the interface structure.

The sheet of flexible material 1002 is configured to accommodate movement of the drive assembly interface elements and the instrument interface element to allows the transfer of drive therebetween whilst maintaining the integrity of the sterile barrier throughout a surgical procedure. The flexible material 1002 may be a resilient, low modulus material with good resistance to puncture and tearing. This can permit stretching in the material without the material thereby rupturing. It some cases the flexible material may be a material made of styrenic block copolymers (SBC), such as those sold under the name Kraton® or other similar high performance elastomers.

In some cases, as shown in FIGS. 10 and 11, the sheet of flexible material may comprise one or more drive transfer elements 1008a, 1008b, 1008c. Each drive transfer element 1008a, 1008b, 1008c is configured to transfer drive between a drive assembly interface element and the corresponding instrument assembly interface. Specifically each drive transfer element 1008a, 1008b, 1008c acts as an interface between a drive assembly interface element and the corresponding instrument interface element so that drive can be transferred between the drive assembly interface element and the instrument interface element. FIGS. 10 and 11 show three drive transfer elements, however it will be evident to a person of skill in the are that there may be fewer than three drive transfer elements or more then three drive transfer elements depending on the number of drive assembly interface element and instrument interface element pairs. Suitably the drive transfer elements are made of a material that is stronger than, or more resilient (e.g. tougher) than, the flexible material so as to ensure that the drive transfer elements do not tear when the instrument interface element is engaged with the corresponding drive assembly interface element so that the drive assembly interface element transfers drive to the instrument assembly interface. In some cases the drive transfer elements are made of plastic.

Where the instrument interface elements and the drive assembly interface elements are linearly displaceable, as described above with respect to FIGS. 4-6, the drive transfer elements may be linearly displaceable with respect the rigid rim 1004. Specifically, the flexible material allows the drive transfer elements to move linearly with the corresponding instrument interface element and drive assembly interface element.

As described above, each drive assembly interface element and instrument interface element pair may comprises a fin and a corresponding socket. For example, each instrument interface element may comprise a fin which is received in a socket of the corresponding drive assembly interface element. In these cases, each drive transfer element may comprise a cup-shaped portion 1010a, 1010b, 1010c wherein the inner portion of the cup receives the fin and the outer portion of the cup engages the socket. Where the instrument interface elements comprise the fin and the drive assembly interface elements comprise the socket as described above with respect to FIGS. 5 and 6, the cup-shaped portion 1010a, 1010b, 1010c may extend downwards from a bottom surface of the flexible material 1002. In contrast, where the instrument interface elements comprise the socket and the drive assembly interface elements comprise the fin the cup-shaped portion 1010a, 1010b, 1010c may extend upwards from a top surface of the flexible material 1002. Reference is now made to FIG. 12 which shows a cross section of an instrument engaged with an interface assembly (without the locking ring), which is in turn engaged with the robot arm along the longitudinal axis of the interface structure 702. It can be seen that the fin 504 of instrument interface element 404 engages the inner surface of the cup-shaped portion 1010b of the drive transfer element 1008b, and the outer surface of the cup-shaped portion 1010b engages the socket 610 of drive assembly interface element 604.

In some cases, each drive transfer element may also comprise a rim portion 1012a, 1012b, 1012c surrounding an upper portion of the cup-shaped portion. The rim portion 1012a, 1012b, 1012c extends from upper portion of the cup-shaped portion in a direction parallel to the movement of the drive assembly transfer elements. The rim portion reduces the strain on the flexible material 1002 at the points the cup-shaped portion engages the flexible material 1002 to reduce the likelihood that the flexible material will tear when the instrument interface element and the corresponding drive assembly interface element are engaged so as to transfer drive therebetween.

The drive transfer elements 1008a, 1008b, 1008c can be attached to the sheet of flexible material 1002 in any suitable way, such as, but not limited to, welding (e.g. hot-gas welding, vibration welding, ultrasonic welding, induction welding, infrared/laser welding, and dielectric welding) and bonding (e.g. by an adhesive, by solvent bonding or by fusion bonding). In some cases, the sheet of flexible material is manufactured with holes or apertures for the drive transfer elements and the drive transfer elements are inserted into the holes or apertures and then they are attached to the sheet of flexible material 1002 using a suitable method, such as those described above.

In some cases the drive transfer elements 1008a, 1008b, 1008c are positioned on the flexible material 1002 so as to be aligned with the drive assembly interface elements when the drive assembly interface elements are in a predetermined position. This allows the drive interface elements to be placed in the predetermined position prior to attaching the drive interface structure 706 to the interface structure 702 to make the mating of the drive transfer elements 1008a, 1008b, 1008c and the corresponding drive assembly interface elements easier.

The rigid rim 1004 is attached to an outer edge 1006 of the sheet of flexible material 1002. In other words, the rigid rim 1004 surrounds the sheet of flexible material 1002. In some cases the sheet of flexible material 1002 is bonded to the rigid rim 1004. The rigid rim 1004 is configured to removably engage the interface structure 702 so that when the drive interface structure is engaged with the interface structure the sheet of flexible material 1002 covers the aperture of the interface structure through which the one or more drive assembly interface elements transfer drive to the one or more instrument interface elements. For example, the base portion 806 may comprise one or more alignment features 850a-850f which engage features 1014a, 1014b, 1014c, 1014d of the drive interface structure 706 so as to align the drive interface structure axially with the aperture of the interface structure 702 to ensure the sheet of flexible material 1002 covers the aperture. In some cases the features 1014a, 1014b, 1014c, 1014d of the drive interface structure 706 may be protrusions formed on an outer edge of the rim 1004. The rigid rim is made of a material that is more rigid than the flexible material. Suitably the rigid rim is made of the same material as the interface structure. In some cases, the rigid rim 1004 is made of plastic.

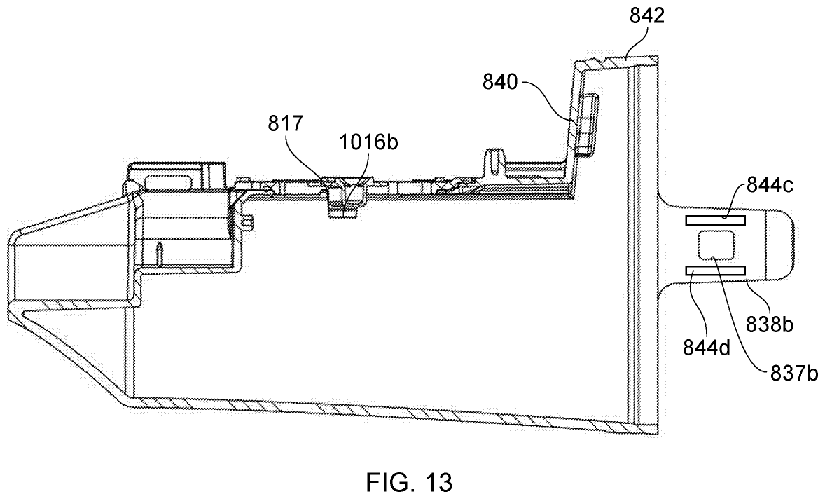

In some cases, as shown in FIG. 11, the drive interface structure 706 may comprise one or more fasteners 1016a, 1016b supported by the rigid rim 1004 and protruding from a bottom surface of the rigid rim 1004. In some cases, the fasteners are configured to engage the surgical robot arm so as to retain the drive interface structure to the surgical robot arm. In other cases, the fasteners are configured to engage the interface structure 702 so as to retain the drive interface structure to the interface structure 702. Suitably the one or more fasteners extend through the aperture of the interface structure when the drive interface structure is engaged with the interface structure 702 (see FIG. 13). In the example shown in FIG. 11 the fasteners 1016a, 1016b are implemented as clips or lugs, but it will be evident to a person of skill in the art that other suitable fasteners may be used.

In some cases, the drive interface structure 706 is configured to engage the interface structure so as to form a seal around the aperture of the interface structure when the drive interface structure is engaged with the interface structure 702. By forming a seal around the aperture, together the drive interface structure 706 and the interface structure 702 provide a complete sterile barrier between the surgical instrument and the surgical robot arm. As shown in FIG. 11, the seal around the aperture may be achieved by having a gasket 1018 on a bottom surface of the rigid rim which is configured to form a seal around the aperture of the interface structure when the drive interface structure is engaged with the interface structure 702. In these cases the gasket 1018 has a shape that is complementary to the shape of the aperture. Alternative the rim of the base portion of the interface structure may comprise a gasket (not shown) that surrounds the aperture and forms a seal around the aperture when the drive interface structure is engaged with the interface structure 702. The gasket 1018 may be formed of a rubber material or any other suitable material that allows a tight seal to be formed between the interface structure 702 and the drive interface structure 706.

In some cases, as shown, in FIGS. 10-11, the rigid rim 1004 comprises one or more protrusions 1014a, 1014b, 1014c, 1014d and 1020 which are configured to engage the surgical instrument when the surgical instrument is brought into engagement with the drive interface structure so as to clamp the seal around the aperture. In other words, when the surgical instrument is brought into engagement with the drive interface structure 706 the surgical instrument pushes down on the one or more protrusions to aid in forming a tight seal around the aperture. In the example shown in FIGS. 10-11 there are four protrusions 1014a, 1014b, 1014c, 1014d situated on each corner of the rigid rim and a single protrusion 1020 that extends along a rear edge 1022 of the rigid rim 1004. Each protrusion has an uppermost surface that engages the surgical instrument when the surgical instrument is brought into engagement with the drive interface structure. It will be evident to a person of the skill in the art that these are example protrusions only and there may be another number (more or fewer) of protrusions, and/or the protrusions may be located elsewhere along the rigid rim 1004.

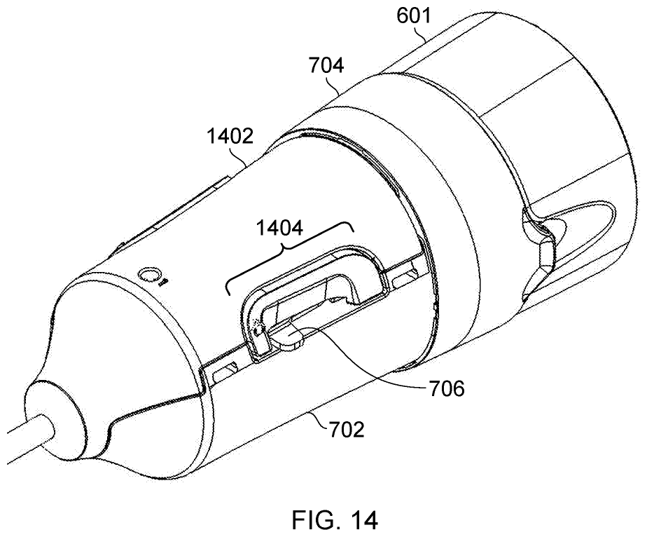

The interface structure 702 may comprise one or more drive interface engagement markings 852a, 852b that are visible when the drive interface structure 706 is not engaged with the interface structure 702 (FIGS. 8-9), and that are not visible when a drive interface structure is fully or properly engaged with the interface structure 702 (FIG. 7, FIG. 14). In some examples, the rigid rim comprises one or more flanges 1026a, 1026b that cover or conceal the drive interface engagement markings when the drive interface structure 706 is engaged with the interface structure 702. The flanges may be integral with the rigid rim 1004. Such markings 852a, 852b provide a visual indication of whether a drive interface structure has been brought into engagement with the interface structure 702. The markings may have a distinct colour, such as, but not limited to, red, with respect to the remainder of the interface structure 702. Having a distinct colour allows the markings to stand out, making it easy for a person near to the robot arm to quickly determine whether a drive interface structure 706 has been brought into engagement with the interface structure 702.

In the example of FIGS. 8-9 there are two drive interface engagement markings 852a, 852b. Each drive interface engagement marking 852a, 852b is situated on the first surface 814 of the base portion 806 between a pair of second fasteners 848a, 848b, 848c, 848d such that when the drive interface structure 706 is completely or properly engaged with the interface structure 702 the drive interface engagement markings 852a, 852b are covered by the flanges 1026a, 1026b of the drive interface structure 706. However, in other examples there may be more than two drive interface engagement markings, or fewer than two drive interface engagement markings, and/or the drive interface engagement marking may be situated in another location so as to be covered by another component of the drive interface structure 706.

Reference is now made to FIG. 14 which illustrates an engaged configuration of a surgical robot arm and a surgical instrument. In the example of FIG. 14, the surgical instrument comprises a body 1402 and two engagement portions 1404 (one shown), all of which cover the instrument interface 400 shown in FIGS. 4 and 5. The engagement portions are on opposing sides of the instrument interface. One engagement portion engages the second fasteners on one side of the instrument interface, and the other engagement portion engages the second fasteners on the other side of the instrument interface. Each engagement portion is displaceable relative to the body in direction A which is transverse to the longitudinal axis 409 of the instrument shaft 402. Each engagement portion 1404 is displaceable along the direction A towards the interior of the instrument.

The exterior body of the instrument 1302 and the exterior body of the robot arm is shaped to be frustoconical when the instrument is docked to the robot arm.

The engagement portion 1404 is biased towards adopting the engaged position. The engagement portion may, for example, be spring-loaded so as to bias its position towards the engaged position.

The interface structure 702 may further include a wireless receiver for receiving wireless transmissions from the surgical instrument. Preferably, the wireless receiver operates according to a communications protocol which has a short range, for example NFC, WiFi or Bluetooth. The receiver receives transmissions relating to the surgical instrument. For example, the receiver may receive transmissions relating to one or more of the following attributes of the surgical instrument: identity, type, origin, status, number of times used, length of time used, number of times left to use before expiry, length of time left to use before expiry. The wireless receiver may be located anywhere on the interface structure. Preferably, it is located on the side of the interface structure which faces the surgical robot arm. The wireless receiver may be a wireless transceiver which also incorporates a transmitter for transmitting wireless transmissions to the surgical instrument.

The surgical instrument may comprise a wireless transmitter for sending wireless transmissions to the interface structure as described above. The surgical instrument may comprise a wireless transceiver for sending and receiving wireless communications as described above.

Suitably, the interface assembly 700 is fastened to the drive assembly as the robot arm is being shrouded in the sterile drape as part of the set-up procedure prior to the operation beginning. Specifically the interface structure 702 (with the locking element 704 mounted thereon) is push fitted onto the robot arm by applying force in the axial direction K (see FIGS. 8-9). This causes the first fasteners 838a, 838b to engage the robot arm. The locking element 704 is then pushed into the locked position so as to lock the interface structure 702 to the robot arm. The drive interface structure 706 is then manually fitted on the interface structure 702 so the flexible material 1002 covers the aperture. Once the interface assembly 700 has been locked onto the arm and the drive interface structure engaged therewith, the drape is unravelled down the robot arm.

At some point during the operation, the instrument may be exchanged for another instrument. The instrument is detached from the interface structure 702 by the user moving the engagement portion 1404 in the direction A until reaching the disengaged position. The user then lifts the instrument off the interface structure in a direction B which is perpendicular to the longitudinal axis 409 of the instrument shaft 402 away from the interface structure. A different instrument can then be attached to the interface structure as previously described.

This method enables the instruments to be quickly and easily detached and attached to the robot arm during an operation without exposing the patient to a non-sterile environment. Since the instrument is removed by lifting it off the robot arm in a direction perpendicular to the shaft 402 of the instrument. Thus, there is no risk of pushing the instrument into the patient when detaching or attaching it.

The first fasteners depicted in the figures have the form of a clip for clipping into the recesses of the robot arm. The second fasteners depicted in the figures have the form of a socket for receiving a plug/nib from the instrument. However, the fasteners may take any suitable form, for example clips, clasps, buckles, latches, plugs, sockets, hooks, eyes, poppers, eyelets, buttons, Velcro, as long as the following criteria are satisfied:

1) the interface structure remains attached to the robot arm when the instrument is detached.

2) the interface structure, instrument and robot arm do not shift relative to each other along their longitudinal axes as articulation of the instrument is driven by the robot arm.

The instrument could be used for non-surgical purposes. For example it could be used in a cosmetic procedure.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A drive interface structure for interfacing one or more drive assembly interface elements of a surgical robot arm and one or more instrument interface elements of a surgical instrument, the drive interface structure comprising:
a sheet of flexible material for providing a sterile barrier between the one or more instrument interface elements and the one or more drive assembly interface elements; and
a rigid rim attached to an outer edge of the sheet of flexible material so as to surround the sheet of flexible material, the rigid rim configured to removably engage an interface structure for detachably interfacing the surgical robot arm to the surgical instrument so that when the drive interface structure is engaged with the interface structure the sheet of flexible material covers an aperture of the interface structure through which the one more drive assembly interface elements transfer drive to the one or more instrument interface elements;
wherein the sheet of flexible material is configured to accommodate movement of the one or more drive assembly interface elements and the one or more instrument interface elements to allow transfer of drive thereby while maintaining the sterile barrier.

2. The drive interface structure of claim 1, wherein the flexible material comprises one or more drive transfer elements for transferring drive between the one or more instrument interface elements and the one or more drive assembly interface elements.

3. The drive interface structure of claim 2, wherein the one or more drive transfer elements are linearly displaceable with respect the rigid rim.

4. The drive interface structure of claim 36, wherein each of the one or more drive transfer elements comprises a rim portion surrounding the cup-shaped portion.

5. The drive interface structure of claim 2, wherein the one or more drive transfer elements are formed of a more resilient material than the flexible material.

6. The drive interface structure of claim 1, further comprising one or more fasteners supported by the rigid rim and protruding from a bottom surface of the rigid rim, the one or more fasteners configured to engage the surgical robot arm or the interface structure so as to retain the drive interface structure to the surgical robot arm.

7. The drive interface structure of claim 6, wherein the one or more fasteners are configured to extend through the aperture of the interface structure when the drive interface structure engages the interface structure.

8. The drive interface structure of claim 1, further comprising a gasket on a bottom surface of the rim, the gasket configured to form a tight seal around the aperture when the drive interface structure engages the interface structure.

9. The drive interface structure of claim 1, wherein the rigid rim is configured to engage a gasket surrounding the aperture so as to form a tight seal around the aperture when the drive interface structure engages the interface structure.

10. The drive interface structure of claim 8, wherein the rigid rim comprises one or more protrusions which are configured to engage the surgical instrument when the surgical instrument is brought into engagement with the drive interface structure so as to clamp the seal around the aperture when the drive interface structure engages the interface structure.

11. The drive interface structure of claim 10, wherein at least one of the one or more protrusions is situated on a corner of the rigid rim.

12. The drive interface structure of claim 10, wherein at least one of the one or more protrusions extends along a rear edge of the rigid rim.

13. The drive interface structure of claim 1, wherein the rigid rim comprises one or more flanges configured to conceal one or more drive interface engagement markings on the interface structure when the drive interface structure is engaged with the interface structure.

14. (canceled)

15. An interface structure for detachably interfacing a surgical robot arm to a surgical instrument, the interface structure comprising:
a base portion comprising a rim surrounding an aperture through which one or more drive assembly interface elements of the surgical robot arm transfer drive to one or more instrument interface elements of the surgical instrument, the rim comprising a first surface for facing the surgical instrument and a second surface for facing the surgical robot arm; and
one or more protrusions supported by the base portion and protruding from the first surface, the one or more protrusions configured to receive a drive interface structure comprising a sheet of flexible material and align the drive interface structure so that the flexible material of the drive interface structure covers the aperture.

16. The interface structure of claim 15, wherein the first surface comprises one or more drive interface engagement markings that are visible when the drive interface structure is not engaged with the interface structure and that are visible when the drive interface structure is engaged with the interface structure.

17. The interface structure of claim 15, further comprising a plurality of fasteners supported by the base portion and protruding from the first surface, the plurality of fasteners configured to engage the surgical instrument so as to retain the interface structure to the surgical instrument.

18. The interface structure of claim 15, further comprising an envelope portion which connects opposing edges of the base portion so as to, when engaged on the surgical robot arm, retain the interface structure to the surgical robot arm.

19. An interface assembly comprising:
a drive interface structure for interfacing one or more drive assembly interface elements of a surgical robot arm and one or more instrument interface elements of a surgical instrument, the drive interface structure comprising:
a sheet of flexible material for providing a sterile barrier between the one or more instrument interface elements and the one or more drive assembly interface elements; and
a rigid rim attached to an outer edge of the sheet of flexible material so as to surround the sheet of flexible material; and
an interface structure for detachably interfacing the surgical robot arm to the surgical instrument, the interface structure comprising:
a base portion comprising a rim surrounding an aperture through which the one or more drive assembly interface elements transfer drive to the one or more instrument interface elements, the rim comprising a first surface for facing the surgical instrument and a second surface for facing the surgical robot arm; and
one or more protrusions supported by the base portion and protruding from the first surface, the one or more protrusions configured to receive the drive interface structure and align the drive interface structure so that the sheet of flexible material covers the aperture;
wherein the sheet of flexible material is configured to accommodate movement of the one or more drive assembly interface elements and the one or more instrument interface elements to allow transfer of drive thereby while maintaining the sterile barrier.

20. A surgical robot arm for use in robotic surgery, the surgical robot arm comprising:
a base; and
a series of articulations connecting the base to an interfacing portion at the distal end of the surgical robot arm, the series of articulations enabling the interfacing portion to be articulated relative to the base; and
the interfacing portion configured to interface a surgical instrument by retaining the interface assembly as set forth in claim 19.

21.-35. (canceled)

36. The drive interface structure of claim 2, wherein each of the one or more drive transfer elements comprises a cup-shaped portion for receiving a drive assembly interface element or an instrument interface element.
